(19) United States
(12) Patent Application Publication
(10) Pub. No.: US 2025/0082294 A1
(43) Pub. Date: Mar. 13, 2025

(54) X-RAY IMAGING SYSTEM AND DOSE DISPLAY METHOD

(71) Applicant: SHIMADZU CORPORATION, Kyoto-shi (JP)

(72) Inventors: Shohei OKUBO, Kyoto-shi (JP); Takanori YOSHIDA, Kyoto-shi (JP)

(21) Appl. No.: 18/726,837

(22) PCT Filed: Jan. 25, 2023
(86) PCT No.: PCT/JP2023/002227

§ 371 Date: Jul. 5, 2024

(30) Foreign Application Priority Data
Feb. 3, 2022 (JP) 2022-015571

(51) Int. Cl. A61B 6/00; A61B 6/46

(52) U.S. Cl. CPC A61B 6/542; A61B 6/4441; A61B 6/463; A61B 6/465; A61B 6/467; A61B 6/547

(57) ABSTRACT

This X-ray imaging system is equipped with a dose calculation processing unit and a display unit. As described above, the dose calculation unit calculates the dose in each of a plurality of angular regions partitioned at each predetermined angular interval of the imaging unit angle, based on the dose distribution on the surface of the virtual model and the imaging unit angle associated with the surface of the virtual model. The display unit is configured to display an angular dose image capable of identifying the magnitude of the calculated doses in each of the plurality of angular regions.

### TECHNICAL FIELD

The present invention relates to an X-ray imaging system and a dose display method for displaying an X-ray dose.

### BACKGROUND ART

Conventionally, an X-ray imaging apparatus that displays an X-ray dose is known. Such a device is disclosed, for example, in U.S. Patent Application Publication No. 2011/0317815.

The X-ray imaging apparatus described in U.S. Patent Application Publication No. 2011/0317815 displays a three-dimensional model that visualizes the dose of X-rays irradiated on the surface of the patient. Specifically, this X-ray imaging apparatus calculates the dose of X-rays irradiated on the surface of the patient on a three-dimensional model of the patient. The magnitude of the calculated dose is then visualized on the displayed three-dimensional model.

### PRIOR ART DOCUMENT

### Patent Document

Patent Document 1: International Publication No. WO 2011/0317815

### SUMMARY OF THE INVENTION

### Problems to be Solved by the Invention

Conventionally, an X-ray imaging apparatus, such as the one described in U.S. Patent Application Publication No. 2011/0317815, is used in interventional radiology (IVR). When interventional radiology treatment is performed while emitting X-rays, it is necessary to perform the treatment while changing the angle of the imaging unit according to the increase in dose to prevent a localized increase in the dose (skin dose) on the body surface of the subject (patient). However, in the case where the magnitude of the dose is displayed on the three-dimensional model, as in U.S. Patent Application Publication No. 2011/0317815, even if the dose displayed on the three-dimensional model is confirmed, it is not possible to concretely determine the appropriate imaging unit angle to avoid positions of relatively high dose (an angle with a relatively low dose). Therefore, it is desirable to easily select an appropriate imaging unit angle with a relatively low dose in order to suppress the localized increase in dose on the body surface of the subject.

The present invention has been made to solve the above-described problems. One object of the present invention is to provide an X-ray imaging system and a dose display method capable of easily selecting an appropriate imaging unit angle with a relatively small dose to suppress the localized increase in dose on the body surface of the subject.

### Means for Solving the Problems

In order to attain the above-described object, an X-ray imaging system according to the first aspect of the present invention comprises:
an imaging unit including an X-ray irradiation unit having an X-ray source that irradiates a subject on a table with X-rays and an X-ray detection unit that detects X-rays emitted from the X-ray irradiation unit;
a moving mechanism configured to change an imaging unit angle which is an angle of the imaging unit;
a dose calculation processing unit configured to calculate a dose of X-rays irradiated onto the subject; and
a display unit configured to display the dose of X-rays calculated by the dose calculation processing unit,
wherein the dose calculation processing unit includes
a model dose calculation unit configured to calculate a dose distribution on a surface of a three-dimensional virtual model representing the subject on the table, based on the dose of the X-rays irradiated onto the subject by the imaging unit,
an angle associating unit configured to associate the imaging unit angle with a surface of the virtual model, based on the imaging unit angle and a position on the surface of the virtual model to be irradiated with X-rays, and,
an angular dose calculation unit configured to calculate the dose in each of a plurality of angular regions partitioned by each of predetermined angular intervals of the imaging unit angle, based on the dose distribution on the surface of the virtual model calculated by the model dose calculation unit and the imaging unit angle associated with the surface of the virtual model by the angle associating unit, and
wherein the display unit is configured to display an angular dose image capable of identifying magnitude of the dose in each of the plurality of angular regions calculated by the angular dose calculation unit.

A dose display method according to the second aspect of the present invention comprise:
a step of calculating a dose distribution on a surface of a three-dimensional virtual model representing a subject on a table, based on the dose of X-rays irradiated onto the subject by X-ray irradiation by an imaging unit, the imaging unit including an X-ray irradiation unit having an X-ray source that irradiates the subject on the table with X-rays and an X-ray detection unit that detects the X-rays irradiated from the X-ray irradiation unit;
a step of associating an imaging unit angle with a surface of the virtual model, based on the imaging unit angle that is an angle of the imaging unit and a position on a surface of the virtual model to be irradiated with the X-rays;
a step of calculating the dose in each of a plurality of angular regions partitioned by each of predetermined angular intervals of the imaging unit angle, based on the calculated dose distribution on the surface of the virtual model and the imaging unit angle associated with the surface of the virtual model; and
a step of displaying an angular dose image capable of identifying magnitude of the calculated dose in each of the plurality of angular regions.

### Effects of the Invention

The X-ray imaging system according to the first aspect of the present invention and the dose display method according to the second aspect of the present invention calculate the dose in each of a plurality of angular regions partitioned at each predetermined angle interval of the imaging unit angle, based on the dose distribution on the surface of the virtual model calculated and the imaging unit angles associated with the surface of the virtual model. The first X-ray imaging system according to the first aspect of the present invention and the dose display method according to the second aspect of the present invention display an angular dose image capable of identifying the magnitude of the dose in each of the plurality of angular regions. With this, an operator (surgeon), such as a doctor, can easily determine the magnitude of the dose in each of the plurality of angular regions partitioned at each predetermined angular interval of the imaging unit angle by visually confirming the displayed angular dose image, which enables the operator to easily determine the imaging unit angle with a relatively small dose. As a result, it is possible to easily select an appropriate imaging unit angle with a relatively low dose in order to suppress the localized increase in dose on the body surface of the subject.

### BRIEF DESCRIPTION OF THE DRAWINGS

FIG. 1 is a schematic diagram for explaining the configuration of the X-ray imaging system.

FIG. 2 is a block diagram showing an entire configuration of the X-ray imaging system according to the embodiment.

FIG. 3 is a diagram for explaining the display of the monitor according to the embodiment.

FIG. 4 is a block diagram for explaining the functional configuration of a dose calculation processing unit according to the embodiment.

FIG. 5 is a diagram for explaining the acquisition of the positional relation between the table and the imaging unit.

FIG. 6 is a diagram for explaining the calculation of the dose distribution in the virtual model.

FIG. 7 is a diagram for explaining the association between the surface of the virtual model and the imaging unit angles.

FIG. 8 is a diagram for explaining the angular dose image and the color scale image.

FIG. 9 is a diagram for explaining updating of the angular dose image when the table is moved vertically with respect to the subject.

FIG. 10 is a diagram for explaining the updating of the angular dose image when the table is moved in the left-right direction with respect to the subject.

FIG. 11 is a diagram for explaining the updating of the angular dose image when the table is moved in the vertical direction.

FIG. 12 is a diagram for explaining the updating of the angular dose image when the angular interval has been changed.

FIG. 13 is an example showing one example of a display of a recommended angular region.

FIG. 14 is a diagram showing one example of a timeline display.

FIG. 15 is a diagram showing one example of a display for selecting an imaging unit angle on a touch panel.

FIG. 16 is a diagram showing a display of an angular dose image on a touch panel.

FIG. 17 is a diagram showing one example of a display of a three-dimensional image.

FIG. 18 is a flowchart for explaining the control processing of the dose display method according to this embodiment.

### EMBODIMENTS FOR CARRYING OUT THE INVENTION

Hereinafter, some embodiments in which the present invention is embodied will be described based on the attached drawings.

### Configuration of X-Ray Imaging System

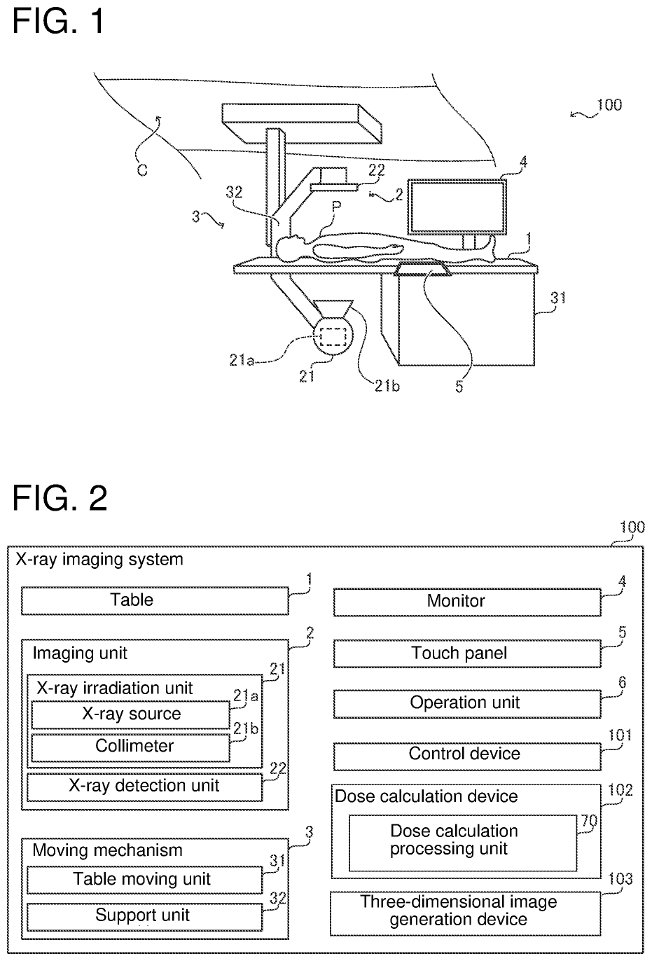

Referring to FIG. 1 to FIG. 17, the configuration of an X-ray imaging system 100 according to one embodiment of the present invention will be described.

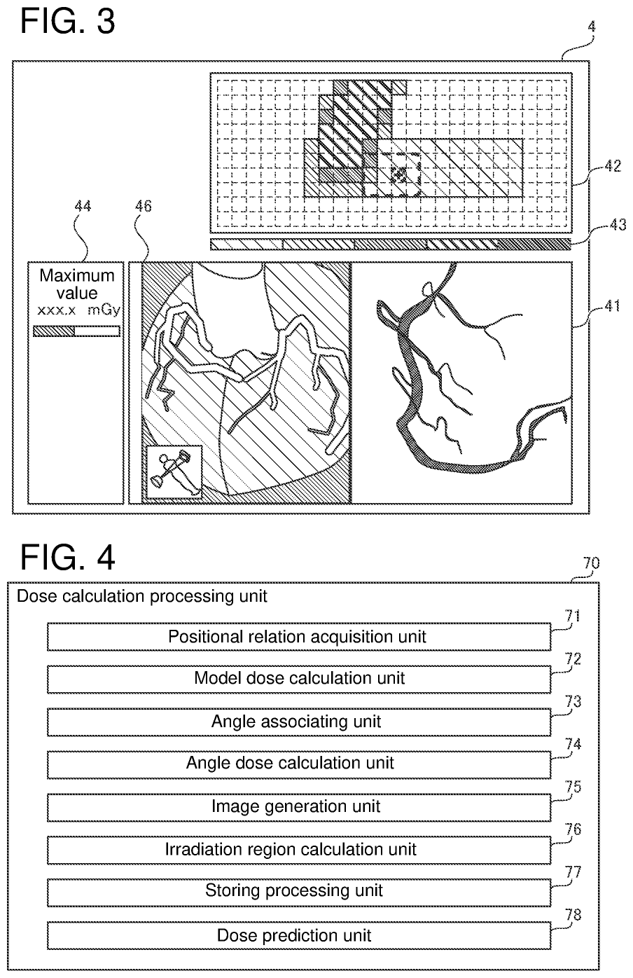

As shown in FIG. 1, the X-ray imaging system 100 is an apparatus that captures X-ray image 41 (see FIG. 3) of the inside of a subject P, such as a human body, by irradiating the subject P with X-rays from the outside of the subject. An operator, such as a doctor, using the X-ray imaging system 100 can perform various types of treatments (interventional radiology: IVR) by inserting a therapeutic device, such as a catheter, into the blood vessel of the subject P (for example, a blood vessel in the heart of the subject P) while visually confirming the X-ray image 41 of the subject P. Note that in this specification, the term “operator” refers not only to the operator who performs the treatment of the subject P but also to an operator who is not directly involved in the treatment of the subject P but simply operates the X-ray imaging system 100. In other words, the operator is one example of the “operator” recited in claims.

As shown in FIG. 1 and FIG. 2, the X-ray imaging system as shown in FIG. 1 and FIG. 2, the X-ray imaging system 100 is equipped with a table 1, an imaging unit 2, a moving mechanism 3, a monitor 4, a touch panel 5, and an operation unit 6. Note that the monitor 4 is one example of the “display unit” according to claims. Further, the touch panel 5 is one example of the “touch panel” recited in claims and serves as an example of the “operation unit” recited in claims.

### Configuration of Each Part of X-Ray Imaging System

As shown in FIG. 1 and FIG. 2, the table 1 is configured as an examination table on which the subject P lies (is placed) when performing X-ray imaging. The table 1 is supported by the table moving unit 31, which will be described later, in a movable manner in the horizontal direction and in the vertical direction. Further, the table 1 is configured to allow the angle of the mounting surface to be changed while the subject P is on it, by means of the table moving unit 31, which will be described later.

As shown in FIG. 1 and FIG. 2, the imaging unit 2 captures an X-ray image 41 (see FIG. 3) of the subject P. Further, the imaging unit 2 includes an X-ray irradiation unit 21 and an X-ray detection unit 22. The X-ray irradiation unit 21 and the X-ray detection unit 22 are arranged so that they face each other across the table 1 on which the subject P lies. The X-ray irradiation unit 21 and the X-ray detection unit 22 are movably supported by the support portion 32 that will be described later.

The X-ray irradiation unit 21 includes an X-ray source 21a and a collimator 21b. The X-ray source 21a irradiates the subject P on the table 1 with X-rays. Further, the X-ray source 21a is an X-ray tube connected to a high-voltage generation unit, not shown in the figure, that generates X-rays when a high-voltage is applied and irradiates the subject P with the generated X-rays. The X-ray source 21a is arranged such that the X-ray emission direction faces the detection surface of the X-ray detection unit 22. The collimator 21b is configured to adjust the irradiation field of the X-rays emitted by the X-ray source 21a. The X-ray irradiation unit 21 generates X-rays according to preset imaging conditions, such as a tube voltage, a tube current, and an X-ray irradiation time interval, under the control of the control device 101, which will be described later.

The X-ray detection unit 22 detects the X-rays emitted from the X-ray irradiation unit 21. The X-ray detection unit 22 outputs a detection signal corresponding to the detected X-ray intensity. The X-ray detection unit 22 is configured, for example, with an FPD (Flat Panel Detector). The X-ray detection unit 22 is connected to the control device 101, which will be described later.

The moving mechanism 3 moves at least one of the table 1 and the imaging unit 2. Specifically, the moving mechanism 3 changes the relative positional relation between the table 1 and the imaging unit 2, thereby changing the position of the body surface of the subject P to be irradiated with X-rays. Specifically, the moving mechanism 3 includes a table moving unit 31 that moves the table 1 and a support unit 32 that changes the imaging unit angle of the imaging unit 2. The table moving unit 31 is configured to move the table 1 in the horizontal direction (the direction parallel to the horizontal plane) and in the vertical direction. Further, the table moving unit 31 changes the angle of the table 1. Further, the support unit 32 is mounted on the ceiling C to support the X-ray irradiation unit 21 and the X-ray detection unit 22 so that they face each other across the table 1 on which the subject P lies. And, the support unit 32 supports the imaging unit 2 in a manner that allows its position and angle (imaging unit angle) to be changed. Further, the support unit 32 supports the imaging unit so that the distance between the X-ray irradiation unit 21 and the X-ray detection unit 22 can be changed. The moving mechanism 3 includes, for example, a servo motor controlled by the control device 101, which will be described later.

For example, when a catheter treatment of a heart is performed by imaging radiotherapy (IVR), the treatment is performed while changing the X-ray irradiation angle (imaging unit angle) to suppress the localized increase in dose on the body surface (skin) of the subject P while continuously acquiring the fluoroscopic image (X-ray image 41) of the heart as a moving image. In this embodiment, the moving mechanism 3 is configured to change the imaging unit angle, which is the angle of the imaging unit 2, through the control processing by the control device 101, which will be described later. The imaging unit angle is an angle in the direction along which the X-ray irradiation unit 21 and the X-ray detection unit 22 face each other. For example, the imaging unit 2 changes the angle of the imaging assembly to enable the X-ray detection unit 22 to move in the LAO (left anterior oblique) and RAO (right anterior oblique) directions, representing the leftward and rightward directions of the subject P, as well as in the CRA (Cranial) and CAU (Caudal) directions, representing the upward and downward directions (head side and leg side), based on the reference state where the X-ray irradiation unit 21 and the X-ray detection unit 22 are aligned vertically.

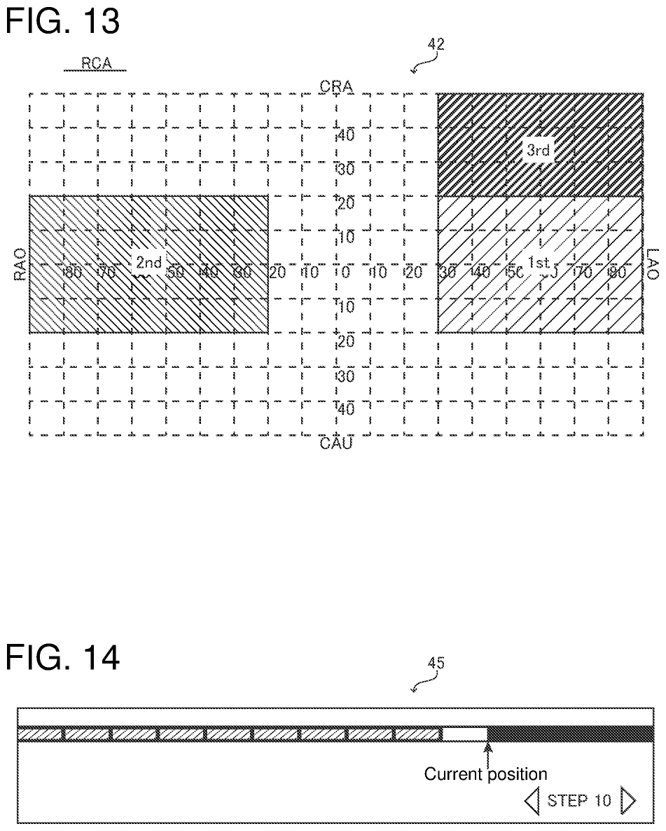

As shown in FIG. 3, the monitor 4 displays an X-ray image 41, an angular dose image 42, a color scale image 43, a maximum value display 44, a timeline display 45 (see FIG. 14), and a three-dimensional image 46, all based on the control by the control device 101, which will be described later. The monitor 4 includes, for example, a display monitor, which is a device that displays video signals of still or moving images output from a device such as a computer. The details of the display on the monitor 4 will be described later.

Further, as shown in FIG. 1 and FIG. 2, the touch panel 5 accepts an input operation by an operator (surgeon or operator) to operate the X-ray imaging system 100. Further, the touch panel 5 is configured to transmit the input signal based on the accepted input operation to the control device 101 and the dose calculation device 102, which will be described later. Specifically, the touch panel 5 is a tablet PC equipped with an operating unit such as a CPU (Central Processing Unit) and a storage unit such as flash memory, and is configured to be able to communicate with the control device 101 and the dose calculation device 102, which will be described later. Further, the touch panel 5 is configured to display information regarding the selection of the imaging unit angle. Further, the details of the display on the touch panel 5, will be described later.

Further, as shown in FIG. 1 and FIG. 6, the touch panel 5 accepts an input operation by an operator (surgeon or operator). The operation unit 6 includes, for example, a pointing device, such as a keyboard and a mouse. Further, the operation unit 6 includes an irradiation button that accepts an input operation for X-ray irradiation and a moving operation button that accepts an input operation for moving the table moving unit 1 and the imaging unit 2. Further, the input unit 6 is configured to transmit an input signal based on the accepted input operation to the control device 101, which will be described below.

In other words, the operation of each part of the X-ray imaging system 100 is controlled based on the input operation to the touch panel 5 and the operation unit 6. For example, the touch panel 5 and the operation unit 6 accept an input operation to select (change) an imaging unit angle for X-ray imaging from a plurality of imaging unit angles. Further, the touch panel 5 and the operation unit 6 accept an input operation to change the display on the monitor 4. Further, the touch panel 5 and the operation unit 6 accept an input operation for performing X-ray irradiation.

Further, as shown in FIG. 2, the X-ray imaging system 100 is equipped with a control device 101, a dose calculation device 102, and a three-dimensional image generation device 103.

The control device 101 is a computer (computing device) configured to include a CPU, a GPU (Graphics Processing Unit), a ROM (Read Only Memory), and a RAM (Random Access Memory). Further, the control device 101 includes a storage device, such as an HDD (Hard Disk Drive) and an SSD (Solid State Drive). The control device 101 controls the operation of the X-ray imaging system 100 based on the operation by the operator. Further, the control device 101 controls the display of the monitor 4. In other words, the control device 101 controls X-ray imaging by controlling the imaging unit 2 to generate the X-ray image 41. Specifically, the control device 101 controls the operation of the X-ray irradiation unit 21 to emit X-rays to the subject P. Further, the control device 101 acquires the detection signal output from the X-ray detection unit 22 and generates the X-ray image 41 based on the acquired detection signal.

As shown in FIG. 3, the X-ray image 41 is an image representing the inside of the subject P based on the detection signal of X-rays transmitted through the subject P. The X-ray image 41 is acquired continuously so that the operator can observe the inside of the subject P in real time. The X-ray image 41 is configured to be displayed on the monitor 4 as a moving image.

Further, the control device 101 changes the relative position and angle between the table 1 and the imaging unit 2 by controlling the operation of the moving mechanism 3, based on the input operations to the touch panel 5 and the operation unit 6. In other words, the control device 101 is configured to set at which imaging unit angle to perform X-ray imaging by controlling the operation of the moving mechanism 3.

The dose calculation device 102 is equipped with a dose calculation processing unit 70, which is a computer (operation unit) including a CPU, a GPU, a ROM, a RAM, etc. Further, the dose calculation device 102 is equipped with a storage device, such as an HDD and an SSD, that stores predetermined programs for operating the dose calculation processing unit 70. The storage device stores a virtual model Pa, which will be described later, various set values (parameters), etc. Further, the storage device stores the history of dose irradiated onto the subject P. In this embodiment, the dose calculation processing unit 70 is configured to calculate the dose of X-rays irradiated onto the subject P.

The three-dimensional image generation device 103, like the control device 101 and the dose calculation device 102, is a computer (computing device) that includes a CPU, a GPU, a ROM, a RAM, etc. The three-dimensional image generation device 103 generates a three-dimensional image 46. The details of the generation of the three-dimensional image 46 will be described later.

In the X-ray imaging system 100, the control device 101, the dose calculation device 102, and the three-dimensional image generation device 103 are configured to send and receive signals to and from each other. The control device 101, the dose calculation device 102, and the three-dimensional image generation device 103 are connected to each other via a computer network, such as, for example, a Local Area Network (LAN).

### Display of Skin Dose

As shown in FIG. 3, in this embodiment, the monitor 4 displays the X-ray dose calculated by the dose calculation device 102. Specifically, the monitor 4 is configured to display the angular dose image 42 generated by the dose calculation device 102. Specifically, the angular dose image 42 generated by the dose calculation device 102 is transmitted to the control device 101. And, the control device 101 displays the angular dose image 42 on the monitor 4.

As shown in FIG. 4, the dose calculation processing unit 70 of the dose calculation device 102 includes, as functional configurations, a positional relation acquisition unit 71, a model dose calculation unit 72, an angle associating unit 73, an angular dose calculation unit 74, an image generation unit 75, an irradiation region calculation unit 76, a storing processing unit 77, and a dose prediction unit 78. That is, the dose calculation processing unit 70 functions as the positional relation acquisition unit 71, the model dose calculation unit 72, the angle associating unit 73, the angular dose calculation unit 74, the image generation unit 75, the irradiation region calculation unit 76, the storing processing unit 77, and the dose prediction unit 78 by executing the prescribed programs stored in the storage device.

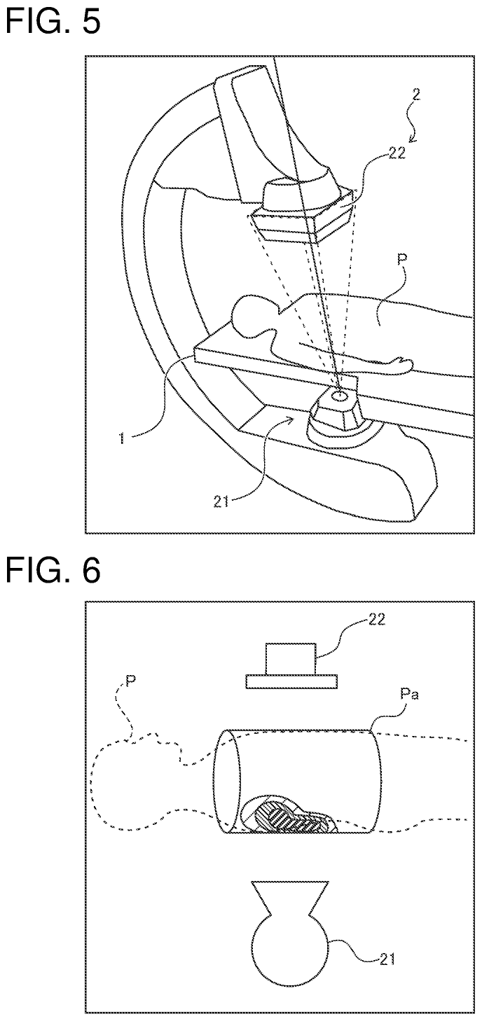

As shown in FIG. 5, in this embodiment, the positional relation acquisition unit 71 (dose calculation processing unit 70) is configured to acquire a virtual positional relation between the virtual model Pa and the imaging unit 2, based on the positional relation between the table 1 and the imaging unit 2.

Specifically, the dose calculation device 102 is configured to acquire the position and angle of the table 1 and the position and angle of the imaging unit 2 from the control device 101. The positional relation acquisition unit 71 then acquires the positional relation between the table 1 and the imaging unit 2, in the three-dimensional virtual space, based on the acquired position and angle of the table 1 and the position and angle of the imaging unit 2 (the X-ray irradiation unit 21 and the X-ray detection unit 22). Further, the positional relation acquisition unit 71 acquires a virtual model Pa (see FIG. 6) representing the subject P on the table 1 in the three-dimensional virtual space. The virtual model Pa is a three-dimensional model with a cylindrical shape. Further, the virtual model Pa has been stored in advance in the storage device of the dose calculation device 102. Further, the virtual model Pa may be resized according to the body size (BMI: Body Mass Index, etc.) of the subject P. The positional relation acquisition unit 71 then acquires the virtual positional relation between the imaging unit 2 and the virtual model Pa in the three-dimensional virtual space. Note that the position of the virtual model Pa in the three-dimensional virtual space is set based on the position and angle of the table 1.

As shown in FIG. 6, in this embodiment, the model dose calculation unit 72 (the dose calculation processing unit 70) is configured to calculate the dose distribution on the surface of the three-dimensional virtual model Pa, based on the dose of X-rays irradiated onto the subject P by the X-ray irradiation by the imaging unit 2 (the X-ray irradiation unit 21).

Specifically, the dose calculation device 102 acquires the imaging conditions, such as the period of the X-ray irradiation, the imaging unit angle, the tube voltage, the tube current, and the X-ray irradiation time interval, from the control device 101. Further, the dose calculation device 102 acquires the dose of X-rays irradiated onto the subject P and the imaging unit angle in an associated manner, from the control device 101. And, the model dose calculation unit 72 calculates the cumulative value of the dose on the surface of the virtual model Pa, based on the dose of X-rays irradiated onto the subject P, the imaging unit angle, and the virtual positional relation between the imaging unit 2 and the virtual model Pa in the three-dimensional virtual space. Then, the model dose calculation unit 72 updates the cumulative dose on the surface of the virtual model Pa each time X-ray irradiation is performed by the imaging unit 2. In detail, the model dose calculation unit 72 calculates the cumulative dose for each of microelements on the surface of the virtual model Pa, which is partitioned into microelements.

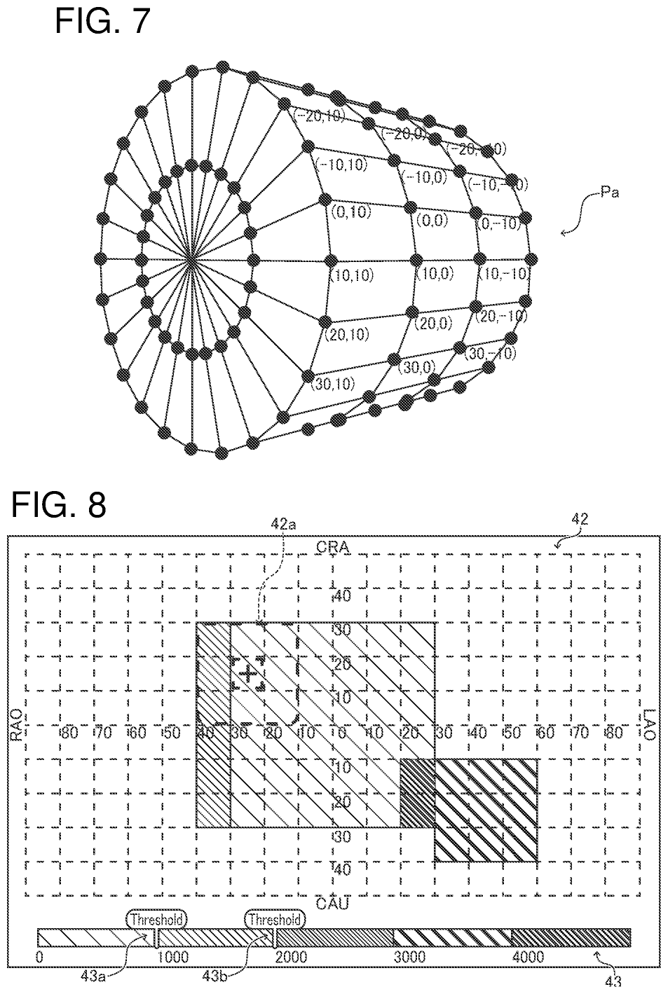

As shown in FIG. 7, in this embodiment, the angle associating unit 73 (the dose calculation processing unit 70) is configured to associate the imaging unit angle with the surface of the virtual model Pa, based on the imaging unit angle and the irradiation position of the X-rays on the surface of the virtual model Pa.

Specifically, the angle associating unit 73 acquires the X-ray irradiation position for each imaging unit angle for each predetermined angular interval on the surface of the virtual model Pa, based on the virtual positional relation between the imaging unit 2 and the virtual model Pa in the three-dimensional virtual space. For example, when the predetermined angular interval is every 10 degrees, the angle associating unit 73 acquires the imaging unit angle when the imaging unit angle is in the vertical direction (reference direction) as (0, 0), the imaging unit angle when the imaging unit angle is tilted by 10 degrees to LAO as (10, 0), and the imaging unit angle when the imaging unit angle is tilted by 20 degrees to LAO as (20, 0). Further, the angle associating unit 73 acquires the imaging unit angle as (10, 10) when tilted by 10 degrees to LAO and also tilted by 10 degrees to CRA. The angle associating unit 73 maps the position of the center (center of the field of view) of the irradiation axis of the X-rays that are irradiated from the imaging unit 2 onto the surface of the virtual model Pa at each predetermined angular interval, based on the positional relation between the imaging unit 2 and the virtual model Pa at each predetermined angular interval (for example, 10 degrees) in the three-dimensional virtual space. The angle associating unit 73 acquires angular regions partitioned on the surface of the virtual model Pa by connecting the position of the center (center of the field of view) of the irradiation axis at each predetermined angular interval partitioned on the surface of the virtual model Pa.

As shown in FIG. 8, in this embodiment, the angular dose calculation unit 74 (dose calculation processing unit 70) is configured to calculate the dose in each of a plurality of angular regions sectioned at each specified angle interval of the imaging unit angle, based on the dose distribution on the surface of the virtual model Pa calculated by the model dose calculation unit 72 and the imaging unit angles that are associated with the surface of the virtual model Pa by the angle associating unit 73. The image generation unit 75 (dose calculation processing unit 70) then generates an angular dose image 42 which can identify the magnitude of the dose in each of the plurality of angular regions calculated by the angular dose calculation unit 74. The angular dose image 42 is an image in which a plurality of angular regions is grated at predetermined angular intervals of the imaging unit angle, and the magnitude of the dose in each of the plurality of angular regions partitioned in a grating pattern can be identified. The angular dose image 42 generated by the image generation unit 75 is output from the dose calculation device 102 to the control device 101 and displayed on the monitor 4.

Specifically, the angular dose calculation unit 74 acquires the maximum value of the dose in each of the angular regions (see FIG. 7) that are partitioned by predetermined angular intervals on the surface of the virtual model Pa by the angle associating unit 73, based on the dose distribution on the surface of the virtual model Pa. And, the image generation unit 75 generates the angular dose image 42 in which each angular region is partitioned into a square at each predetermined angular interval (e.g., 10 degrees), with the LAO and RAO directions as the horizontal axis and the CRA and CAU directions as the vertical axis. In other words, the angular dose image 42 is configured so that the size of the angular interval on the horizontal axis and the size of the angular interval on the vertical axis are of equal size (scale). Further, the ranges of the vertical and horizontal axes are set based on the range of the imaging unit angle that can be moved by the imaging unit 2.

In this embodiment, in the angular dose image 42, the magnitude of the dose in each of the plurality of angular regions is represented by color-coding. For example, in the angular dose image 42, the magnitude of the dose is represented by five different color schemes, from the highest dose to the lowest: purple, red, orange, yellow, and green. Note that in FIG. 8, the differences in color-coding are represented by differences in hatching.

### Color Scale Image

Further, as shown in FIG. 3 and FIG. 8, the monitor 4 is configured to display, separately from the angular dose image 42, the color scale image 43 showing the color-coding of the plurality of angular regions in the angular dose image 42 corresponding to the magnitude of the doses. Further, in this embodiment, the monitor 4 is configured to display preset dose thresholds 43a and 43b in the color scale image 43 in an identifiable manner.

Specifically, the image generation unit 75 generates a color scale image 43 that provides a reference for color coding the angular dose image 42. The color scale image 43 displays the regions that are color-coded to correspond to the color-coding in the angular dose image 42, in the order from the highest to the lowest dose: purple, red, orange, yellow, and green. Further, the boundaries of the five display regions are marked with numerical values indicating the specific dose values that are used as references for color coding. For example, in the color scale image 43, the region where the maximum dose is greater than 4,000 mGy is represented by purple, the region where the maximum dose is greater than 3,000 mGy and less than or equal to 4,000 mGy is represented by red, the region where the maximum dose is greater than 2,000 mGy and less than or equal to 3,000 mGy is represented by orange, the region where the maximum dose is greater than 1,000 mGy and less than or equal to 2,000 mGy is represented by yellow, and the region where the maximum dose is greater than 0 mGy and less than or equal to 1,000 mGy is represented by green.

Further, a display showing the preset dose thresholds 43a and 43b is superimposed on the color scale image 43. The thresholds 43a and 43b are set in advance by a doctor or other operator. Further, it is configured such that a warning message is displayed on the monitor 4 when the dose exceeds the thresholds 43a and 43b in the angular region currently being irradiated in the middle of X-ray irradiation. The warning message is displayed, for example, as a text (character information) at a predetermined portion of the monitor 4. The warning message stops being displayed on the monitor 4 after a predetermined amount of time has elapsed, or by an input operation on the operation unit 6.

It may be configured such that the monitor 4 displays a total skin dose of the subject P during the operation. For example, the dose calculation processing unit 70 calculates the total dose on the entire body surface of the subject P based on the acquired X-ray dose. The total calculated dose is then output from the dose calculation device 102 to the control device 101, and is displayed on the monitor 4 through the control processing by the control device 101.

### Change of Positional Relation

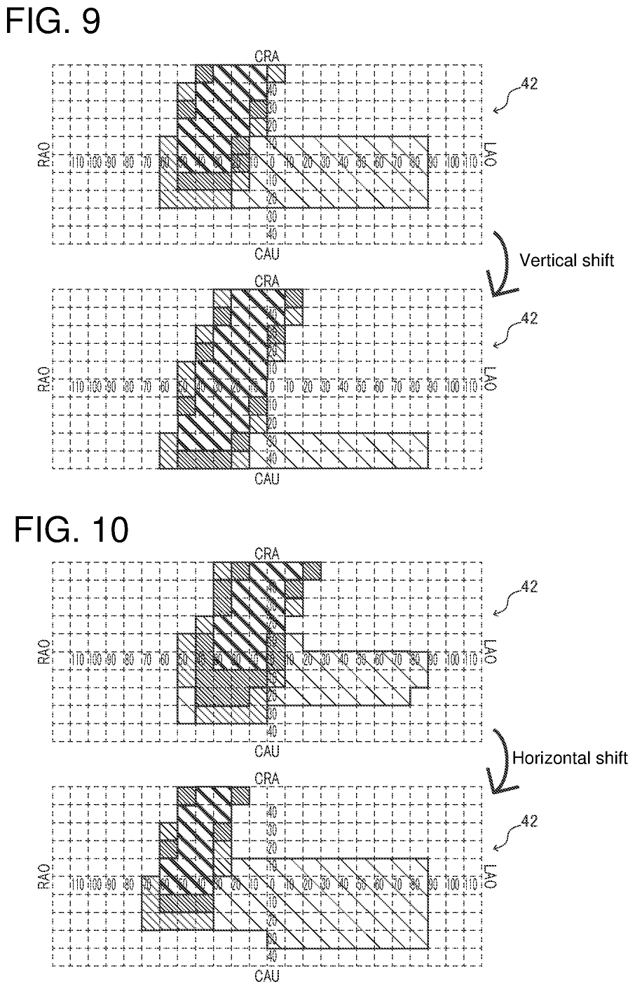

As shown in FIG. 9 to FIG. 11, in this embodiment, the angle associating unit 73 is configured to update the association of the imaging unit angles on the surface of the virtual model Pa, based on the virtual positional relation acquired by the positional relation acquisition unit 71 when the positional relation between the table 1 and the imaging unit 2 is changed. The angular dose calculation unit 74 is configured to update the dose in each of the plurality of angular regions, based on the association of the imaging unit angles on the surface of the updated virtual model Pa. The image generation unit 75 is configured to re-generate the angular dose image 42, based on the updated dose in each of the plurality of angular regions.

Specifically, the positional relation acquisition unit 71 updates the position and angle of the virtual model Pa in the three-dimensional virtual space, based on the information indicating the position and angle of the table 1 newly acquired from the control device 101 when the operation to change the position and angle of the table 1 is accepted. Further, when an operation to change the position and angle of the imaging unit 2 is accepted, the positional relation acquisition unit 71 updates the position and angle of the imaging unit 2 in the three-dimensional virtual space, based on information indicating the position and angle of the imaging unit 2 newly acquired from the control device 101. In other words, the positional relation acquisition unit 71 changes the virtual positional relation between the virtual model Pa and the imaging unit 2 in the three-dimensional virtual space when a signal indicating that the positional relation between the table 1 and the imaging unit 2 has been changed is acquired from the control device 101.

When the virtual positional relation between the virtual model Pa and the imaging unit 2 in the three-dimensional virtual space is changed, the angle associating unit 73 updates the association (mapping) of the imaging unit angle on the surface of the virtual model Pa by newly acquiring (calculating) the X-ray irradiation position for each imaging unit angle for each predetermined angular interval based on the changed virtual positional relation. The angle associating unit 73 acquires angular regions partitioned on the surface of the virtual model Pa by connecting the position of the center (center of the field of view) of the irradiation axis at each predetermined angular interval mapped on the surface of the virtual model Pa.

The angular dose calculation unit 74 newly calculates the dose in each of the plurality of angular regions partitioned at each predetermined angular interval, based on the dose distribution on the surface of the virtual model Pa calculated by the model dose calculation unit 72 and the angular regions by the association of the imaging unit angles updated by the angle associating unit 73. The image generation unit 75 then updates the angular dose image 42 so that the magnitude of the dose in each of the newly recalculated plurality of angular regions can be identified. In other words, when the positional relation between the table 1 and the imaging unit 2 is changed, the degree of the dose distribution in the angular dose image 42 changes based on the dose in each of the plurality of newly recalculated angular regions. Note that in the case of moving (changing) only the imaging unit angle of the imaging unit 2 without moving the table 1, the association of the imaging unit angle on the surface of the three-dimensional virtual model Pa in the three-dimensional virtual space remains unchanged. In other words, in the case of changing only the imaging unit angle, the angular dose image 42 is not updated, and the degree of the dose distribution in the angular dose image 42 remains unchanged.

For example, as shown in FIG. 9, when the table 1 is moved in the longitudinal direction (the vertical direction of the subject P) based on the input operation to the touch panel 5 or the operation unit 6, the degree of dose distribution in the angular dose image 42 moves to shift in the vertical direction (CRA and CAU directions)

Further, as shown in FIG. 10, when the table 1 is moved in the transverse direction (the left-right direction of the subject P) based on the input operation to the touch panel 5 or the operation unit 6, the degree of the dose distribution in the angular dose image 42 moves to shift in the left-right directions (RAO and LAO directions).

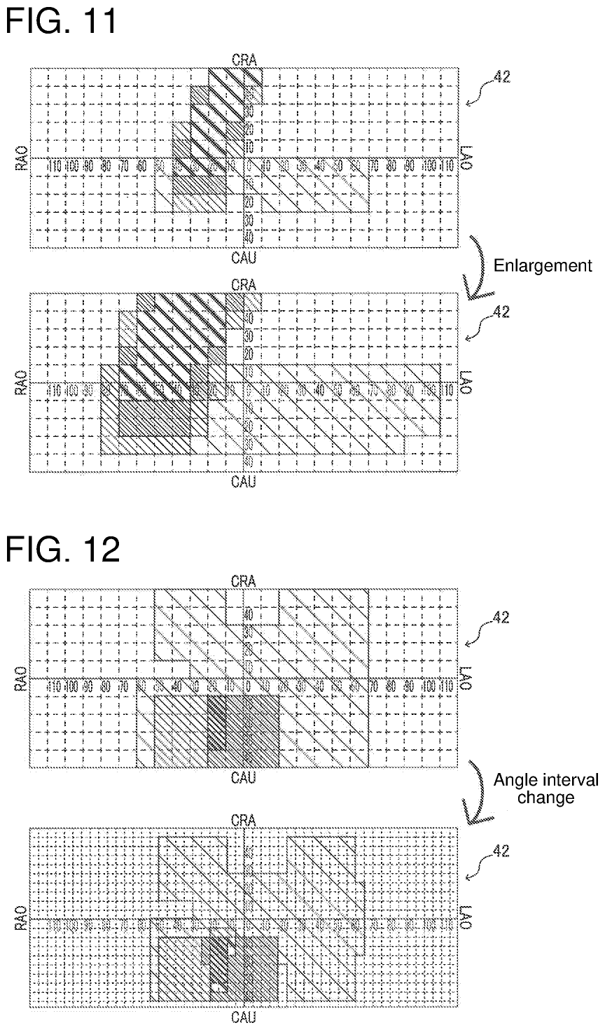

Further, as shown in FIG. 11, when the table 1 is moved in the vertical direction based on the input operation to the touch panel 5 or the operation unit 6, the degree of the dose distribution in the angular dose image 42 changes to be enlarged or reduced.

Similarly, when the angle of the table 1 is changed to rotate it, the degree of the dose distribution in the angular dose image 42 also changes. Further, when the arrangement position of the imaging unit 2 with respect to the table 1 is moved, the degree of the dose distribution in the angular dose image 42 also changes. Note that the change in the degree of the dose distribution in the angular dose image 42 when the positional relation between the table 1 and the imaging unit 2 is changed is different from the simple parallel (shift) moving and enlargement/reduction because the association of the imaging unit angles on the surface of the cylinder-shaped virtual model Pa is changed.

### Change of Angular Interval

Further, as shown in FIG. 12, in this embodiment, the angular dose calculation unit 74 (dose calculation processing unit 70) is configured to calculate the dose in each of the plurality of angular regions partitioned by each of the predetermined angular intervals whose size is changed when an operation to change the size of the predetermined angular interval that partitions the plurality of angular regions is received by the touch panel 5 or the operation unit 6. And, the image generation unit 75 generates an angular dose image 42 capable of identifying the magnitude of the dose in each of a plurality of angular regions where the size of the predetermined angular interval has been changed. The monitor 4 is configured to display an angular dose image 42 in which the size of the predetermined angular interval is changed.

Specifically, when an input operation to change the size of the predetermined angular interval is accepted based on an input operation to the touch panel 5 or the operation unit 6, the angle associating unit 73 reacquires the newly partitioned angular regions on the surface of the virtual model Pa by updating the association (mapping) of the imaging unit angles on the surface of the virtual model Pa so as to be partitioned for each predetermined angular interval whose size has been changed. The angular dose calculation unit 74 recalculates the dose in each of the angular regions whose sizes have been updated. The image generation unit 75 then updates the angular dose image 42 so that the magnitude of the dose in each of the newly recalculated plurality of angular regions can be identified. And, the image generation unit 75 updates the angular dose image 42 so that the magnitude of the dose in each of the newly recalculated plurality of angular regions is identifiable. For example, if the predetermined angular interval is changed from 10 degrees to 5 degrees, the size of the angular dose image 42 itself is not changed, but the size of the partition in the angular dose image 42 is halved. Further, in the angular dose image 42, which is partitioned every 5 degrees, the magnitude of the dose is color-coded and displayed in an identifiable manner based on the distribution of the dose in the angular regions partitioned every 5 degrees on the surface of the virtual model Pa.

### Display of Irradiation Region

As shown in FIG. 8, in this embodiment, the monitor 4 is configured to display a display (the irradiation region display 42a) that shows the angular regions included in the irradiation region from the plurality of angular regions in the angular dose image 42.

Specifically, in this embodiment, the irradiation region calculation unit 76 (dose calculation processing unit 70) is configured to calculate the irradiation region, which is a region of the surface of the virtual model Pa that is irradiated with X-rays at the current imaging unit angle. In detail, the irradiation region calculation unit 76 calculates the irradiation region to be irradiated by X-rays at the current imaging unit angle on the surface of the virtual model Pa, based on the virtual positional relation between the virtual model Pa and the imaging unit 2 in the three-dimensional virtual space acquired by the positional relation acquisition unit 71 and the imaging conditions acquired from the control device 101. Then, the irradiation region calculation unit 76 acquires, from the plurality of angular regions partitioned at predetermined angular intervals by the angle associating unit 73, the angular region that includes inside a part or the entirety of the irradiation region to be irradiated with X-rays, as the angular region included in the irradiation region. The image generation unit 75 superimposes an irradiation region display 42a, which indicates the angular region included in the irradiation region acquired by the irradiation region calculation unit 76, on the angular dose image 42. The angular dose image 42 with the irradiation region display 42a superimposed is displayed on the monitor 4 by being output to the control device 101.

Note that the irradiation region display 42a indicates the irradiation region so as to enclose the entire angular region included in the irradiation region. Further, the irradiation region display 42a may indicate the angular regions included in the irradiation region by coloring them, or by superimposing shaded lines or the like on them. Further, the irradiation region display 42a includes a display capable of identifying the angular regions that include the center of the irradiation axis (center of field of view) of the X-rays to be irradiated from the angular regions included in the irradiation region. For example, the irradiation region display 42a includes a cross (plus shape) mark that indicates the angular region that includes the center (center of field of view) of the irradiation axis in the irradiation region. Further, the irradiation region display 42a changes in size and shape according to the irradiation region (field of view size) of X-rays emitted from the X-ray irradiation unit 21, the aperture of the collimator 21b, or the distance between the X-ray irradiation unit 21 and the X-ray detection unit 22 (SID: source to image distance). The irradiation region display 42a may be not only a square region but also a rectangular or polygonal region, in the angular dose image 42.

Note that, as shown in FIG. 3, in this embodiment, the monitor 4 is configured to display the maximum value of the dose in the angular region included in the irradiation region (the irradiation region display 42a), separately from the angular dose image 42, so as to be identifiable. Specifically, the irradiation region calculation unit 76 is configured to acquire the angular region with the maximum dose from the plurality of angular regions included in the irradiation region. The signal indicating the dose in the angular region where the dose is maximum is output from the dose calculation device 102 to the control device 101. The control device 101 displays, on the monitor 4, the maximum value display 44 showing the maximum value of the dose in the angular region included in the irradiation region (the irradiation region display 42a). In the maximum value display 44, the maximum value from each dose within the plurality of angular regions in the irradiation region (the irradiation region display 42a) is displayed in a state of being colored by the same color-coding in the angular dose image 42. Further, in the maximum value display 44, it may be configured such that a warning message is displayed when the dose exceeds a preset dose (e.g., the threshold 43a).

### Change of Imaging Unit Angle

The X-ray imaging system 100 is configured to change the imaging unit angle, based on the input operation to the touch panel 5 or the operation unit 6. For example, in this embodiment, the operation unit 6 is configured to accept an operation to select one of the plurality of angular regions in the angular dose image 42 displayed on the monitor 4. For example, an operator, such as a doctor, performs a selection operation by clicking on one of the plurality of angular regions partitioned in a grid pattern in the angular dose image 42 displayed on the monitor 4 using the pointer also displayed on the monitor 4. Based on the acceptance of the selection operation by the operation unit 6, the control device 101 changes the imaging unit angle so that X-ray irradiation is performed onto one angular region selected from the plurality of angular regions. In other words, in this embodiment, the moving mechanism 3 is configured to change the imaging unit angle so that the X-rays are irradiated to the selected angular region when an operation to select one of the plurality of angular regions in the angular dose image 42 (selection operation) is received by the operation unit 6.

The irradiation region calculation unit 76 newly acquires the irradiation region display 42a each time the imaging unit angle of the imaging unit 2 is changed. Further, the irradiation region calculation unit 76 is configured to newly acquire the irradiation region display 42a even when the positional relation between the imaging unit 2 and the table 1 is changed. Note that in the X-ray imaging system 100, when only the imaging unit angle of the imaging unit angle is changed, only the irradiation region display 42a is moved (changed), without changing the degree of the dose distribution in the angular dose image 42. Note that in the case of selecting the imaging unit angle in the angular dose image 42, the color-coded display (the colored part) indicating the magnitude of the dose in the angular dose image 42 may be displayed translucently so as to enhance the visibility of the irradiation region display 42a. Further, during the process of changing the imaging unit angle (while moving the imaging unit 2), it may be permissible to display both the moving irradiation region display 42a and the destination irradiation region display 42a in the angular dose image 42.

Further, as shown in FIG. 13, the monitor 4 is configured to display an angular region recommended for X-ray irradiation among the plurality of angular regions in the angular dose image 42 in an identifiable manner. For example, the image generation unit 75 (the dose calculation processing unit 70) colorizes the recommended angular region in the angular dose image 42 to indicate the imaging unit angle at which X-rays are recommended to be irradiated. The recommended imaging unit angle (angular region) display (coloring) is displayed on the monitor 4 when the maximum value of the dose in the angular regions included in the irradiation region (the irradiation region display 42a) at the current imaging unit angle becomes greater than a predetermined dose (e.g., the threshold value 43a).

Further, the recommended imaging unit angle (angular region) is selected based on the site of the subject P to be treated or inspected, based on the information input by the operator in advance, a database stored in the system, or a database acquired through a network. For example, when imaging blood vessels of a heart, the recommended imaging unit angle is preset for each type of blood vessel. In the case where the right coronary artery (RCA: right coronary artery) has been pre-set to be imaged based on an input operation on the touch panel 5 or the operation unit 6, the image generation unit 75 causes the recommended imaging unit angle (angular region), which is set to correspond to the right coronary artery in the angular dose image 42, to be displayed in a color-coded manner. Further, in the case where there is a ranking (priority) of recommended imaging unit angles (angular regions), identification information (character information) for the ranking is also displayed, such as “1^{st}”, “2^{nd}”, and “3^{rd}” in figures. In addition, the image generation unit 75 causes the imaging unit angles (angular regions) corresponding to the respective rankings to be displayed in a color-coded manner so that the rankings are identifiable in the angular dose image 42. In such cases, the recommended imaging unit angle (angular region) may be displayed in such a manner that the high dose regions have already been excluded.

### Timeline Display

As shown in FIG. 14, the monitor 4 displays a timeline display 45 for checking the progress of the irradiated dose. The timeline display 45 is displayed on the monitor 4, for example, based on the input operation to the touch panel 5 or the operation unit 6. The X-ray imaging system 100 is configured to display the angular dose image 42 at a predetermined timing in the past on the monitor 4 based on the selection operation for the timeline display 45.

Specifically, in this embodiment, the storing processing unit 77 (the dose calculation processing unit 70) is configured to store the historical information on the dose distribution on the surface of the virtual model Pa, based on the X-ray irradiation by the imaging unit 2. The storing processing unit 77 makes the storage device of the dose calculation device 102 store the history of the dose of X-rays irradiated each time X-ray irradiation by the imaging unit 2 is performed.

And, the dose calculation processing unit 70 acquires the historical information including the dose value at the selected past predetermined timing from the storage device, based on the operation of selecting one of the timelines in the timeline display 45. Here, in the X-ray imaging system 100, in order to capture X-ray images 41 as moving images, X-ray irradiation is continuously performed over a predetermined period of time in capturing a single X-ray image 41. In the timeline display 45, a predetermined timing in the past is selectably displayed for each predetermined period of time to generate an X-ray image 41 of a single moving image.

The monitor 4 is configured to display the angular dose image 42 at a predetermined timing in the past, based on the historical information stored by the storing processing unit 77. In other words, based on the stored historical information, the dose distribution on the surface of the virtual model Pa and the virtual positional relation between the imaging unit 2 and the virtual model Pa at the selected past predetermined timing are acquired. Then, the same processing as the generation of the angular dose image 42 at the current time is performed to generate the angular dose image 42 based on the dose value at the predetermined timing in the past by the image generation unit 75. The generated angular dose image 42 at a predetermined timing in the past is output to the control device 101 and displayed on the monitor 4. Note that it may be configured such that the storing processing unit 77 causes the generated angular dose image 42 to be directly stored in the storage device at each timing of the generation of the X-ray image 41.

Further, the X-ray imaging system 100 is configured to display the predicted angular dose image 42 on the monitor 4, based on the selection operation for the timeline display 45.

Specifically, in this embodiment, the dose prediction unit 78 (the dose calculation processing unit 70) is configured to calculate the predicted value of the X-ray dose to be irradiated by the imaging unit 2, based on the predictive distribution of the dose on the surface of the virtual model Pa calculated by the model dose calculation unit 72. For example, the dose calculation processing unit 70 accepts a selection of the prediction portion (the portion ahead of the current timing (current position)) of the timeline display 45, based on the input operation to the touch panel 5 or the operation unit 6. The dose prediction unit 78 predicts the increase in dose in the event that X-ray irradiation is continuously performed in the current irradiation region, based on the acceptance of the selection of the prediction portion in the timeline display 45. The dose prediction unit 78 predicts, for example, the increased dose in the case where irradiation is continuously performed, based on the historical information. Further, the dose prediction unit 78 may also predict the increase in dose in the current irradiation region based on a predefined database. Further, the dose prediction unit 78 may also calculate the predicted value of the dose that would be predicted when the imaging condition is changed.

The dose prediction unit 78 then calculates the predicted value of the dose distribution on the surface of the virtual model Pa by calculating the predicted value of the dose. The monitor 4 is configured to display the predicted angular dose image 42 based on the predicted values predicted by the dose prediction unit 78. In other words, the image generation unit 75 generates the angular dose image 42 that is predicted based on the predicted value of the dose distribution on the surface of the virtual model Pa. The generated angular dose image 42 at a predetermined timing in the past is output to the control device 101 and displayed on the monitor 4.

### Details of Touch Panel

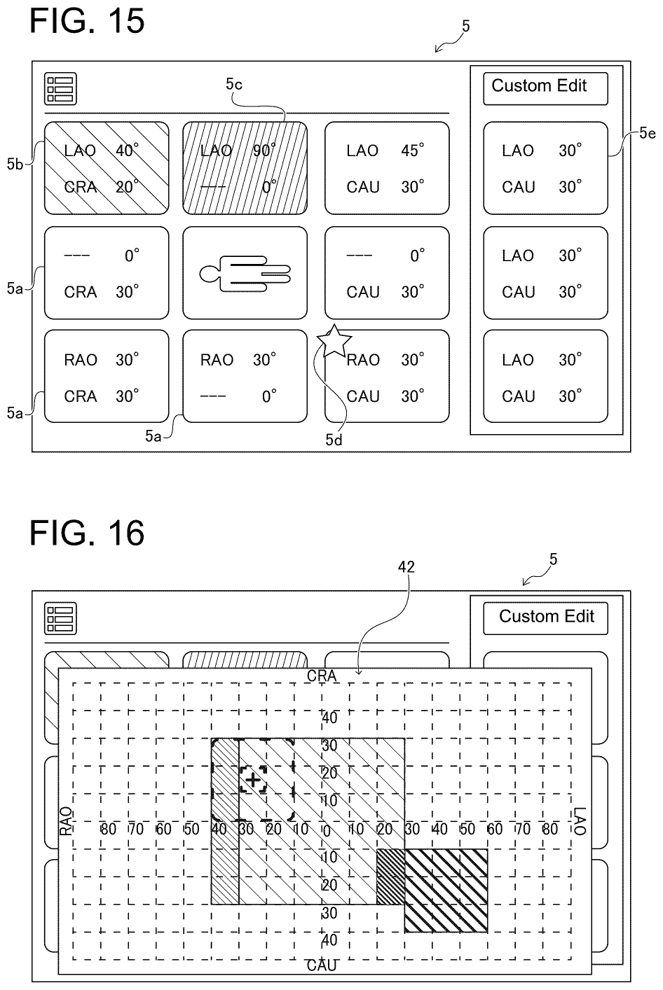

As shown in FIG. 15, in this embodiment, the touch panel 5 displays imaging unit angles in a selectable manner. Further, the touch panel 5 accepts an input operation input to change the imaging unit angle. The touch panel 5 displays a plurality of imaging unit angle displays 5a as a list so that the imaging unit angle can be selected, based on a database stored in a storage unit, such as flash memory included in the touch panel 5, in a storage device of the control device 101, or in a storage device of the dose calculation device 102. Further, the touch panel 5 displays the current angle display 5b, which indicates the current imaging unit angle, in such a manner that it can be identified from the plurality of imaging unit angle displays 5a displayed as a list. Specifically, the touch panel 5 acquires information indicating the position of the imaging unit 2 from the control device 101. The touch panel 5 displays the current imaging unit angle in a visually identifiable manner by displaying the background of the current angle display 5b in blue while displaying the background of the plurality of imaging unit angle displays 5a displayed as a list in white, based on the information acquired from the control device 101.

Further, the touch panel 5 displays a non-recommended angle display 5c indicating a non-recommended imaging unit angle, in a visually identifiable manner, from the plurality of imaging unit angle displays 5a displayed as a list. Specifically, the touch panel 5 acquires the dose calculated for each of the plurality of angular regions from the dose calculation device 102. The touch panel 5 displays the non-recommended angle display 5c so that the imaging unit angles, which are included in the angular regions that exceed a predetermined dose (e.g., threshold 43a), corresponding to the plurality of imaging unit angle displays 5a, can be visually identified as a non-recommended imaging unit angle. For example, the touch panel 5 displays the non-recommended imaging unit angle in a visually identifiable manner by displaying the background of the non-recommended angle display 5c in red.

Further, the touch panel 5 displays a recommended angle display 5d, which is a display (e.g., a star mark) indicating an imaging unit angle at which X-ray irradiation is recommended to be performed. The recommended imaging unit angle is selected based on the site of the subject P to be treated or inspected, based on information input by the operator in advance, a database stored in the system, or a database acquired through a network. Further, the touch panel 5 displays a desired angle display 5e, which indicates the desired imaging unit angle registered by the operator in advance, in a selectable manner. Note that the recommended imaging unit angle and the desired imaging unit angle may be stored in a storage device, such as a flash memory, included in the touch panel 5, in a storage unit of the control device 101, or in a storage device of the dose calculation device 102.

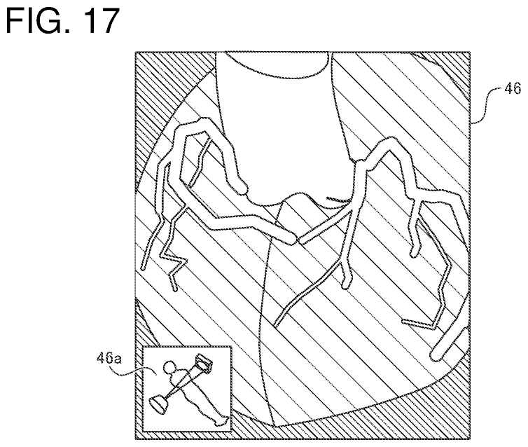

As shown in FIG. 16, the touch panel 5 is configured to display the angular dose image 42. Specifically, the touch panel 5 acquires the angular dose image 42 generated by the image generation unit 75 from the dose calculation device 102. The touch panel 5 is configured to display the acquired angular dose image 42 by, for example, switching the display based on an input operation by the operator.

### Display of Three-Dimensional Image

Further, in this embodiment, as shown in FIG. 3 and FIG. 17, the monitor 4 displays a previously acquired three-dimensional image 46 of the inside of the subject P. Specifically, the three-dimensional image 46 is generated by the three-dimensional image generation device 103. The three-dimensional image generation device 103 generates the three-dimensional image 46 representing the inside of the subject P, based on the three-dimensional image data of the inside of the subject P acquired beforehand by a CT (Computed Tomography: computer tomographic image) device, etc. The three-dimensional image 46 generated by the three-dimensional image generation device 103 is output from the three-dimensional image generation device 103 to the control device 101 to be displayed on the monitor 4.

As shown in FIG. 17, in this embodiment, the monitor 4 is configured to display the dose of the angular region at the imaging unit angle corresponding to the display angle of the displayed three-dimensional image 46 in an identifiable manner. Specifically, the three-dimensional image generation device 103 generates an angle indicator 46a together with the three-dimensional image 46. The angle indicator 46a indicates the display angle of the displayed three-dimensional image 46 by showing the positional relation between the display showing the subject P as the subject and the display showing the imaging unit 2 as the viewpoint so as to correspond to the spatial viewpoint (camera position) of the displayed three-dimensional image 46. The three-dimensional image generation device 103 acquires the dose in each of the plurality of angular regions from the dose calculation device 102 to cause the magnitude of the acquired dose to be displayed on the angle indicator 46a in an identifiable manner. For example, the three-dimensional image generation device 103 will display the indication of the imaging unit 2 in the angle indicator 46a in the same color-coding as in the angular dose image 42. In other words, the three-dimensional image generation device 103 displays the magnitude of the dose in the angular region containing the imaging unit angle corresponding to the current display angle in a color-coded manner in the angle indicator 46a in an identifiable manner. Note that the dose in the angular region containing the imaging unit angle corresponding to the current display angle may be displayed separately from the angle indicator 46a by means of a color-coded icon display or other means similar to the angular dose image 42.

### Control Processing in This Embodiment

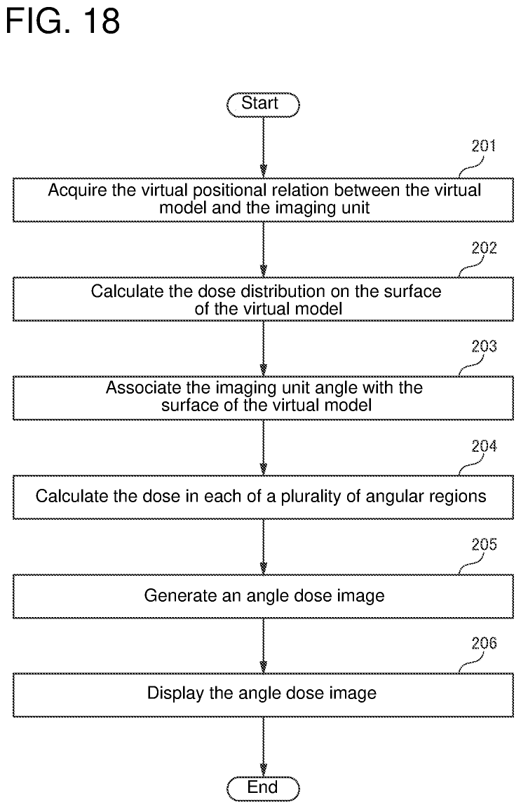

Next, referring to FIG. 18, the control processing flow related to the dose display method by the X-ray imaging system 100 according to this embodiment will be described. Steps 201 to 205 represent the control processing by the dose calculation processing unit 70 (the dose calculation device 102). Further, Step 206 indicates the control processing by the control device 101.

First, in Step 201, the virtual positional relation between the virtual model Pa and the imaging unit 2 is acquired based on the positional relation between the table 1 and the imaging unit 2.

Next, in Step 202, the dose distribution on the surface of the three-dimensional virtual model Pa is calculated based on the dose of X-rays irradiated onto the subject P by the imaging unit 2.

Next, in Step 203, the imaging unit angle is associated with the surface of the virtual model Pa, based on the imaging unit angle and the position where the X-rays are irradiated on the surface of the virtual model Pa.

Next, in Step 204, based on the dose distribution on the surface of the virtual model Pa calculated in Step 202 and the imaging unit angle associated with the surface of the virtual model Pa in Step 203, the dose in each of the plurality of angular regions partitioned at each predetermined angular interval of the imaging unit angle is calculated.

Next, in Step 205, an angular dose image 42 capable of identifying the magnitude of the dose in each of the plurality of calculated angular regions is generated.

Next, in Step 206, the angular dose image 42 is displayed on the monitor 4. Specifically, the generated angular dose image 42 is output from the dose calculation device 102 to the control device 101 and is displayed on the monitor 4. Note that the calculation of the dose distribution on the surface of the virtual model Pa in Step 202 and the association of imaging unit angles with the surface of the virtual model Pa in Step 203 may be performed in either step first.

### Effects of This Embodiment

In this embodiment, the following effects can be obtained.

As described above, the X-ray imaging system 100 calculates the dose in each of a plurality of angular regions partitioned at each predetermined angular interval of the imaging unit angle, based on the calculated dose distribution on the surface of the virtual model Pa and the imaging unit angle associated with the surface of the virtual model Pa. The X-ray imaging system 100 displays the angular dose image 42 capable of identifying the magnitude of the dose in each of the plurality of angular regions. With this, an operator (surgeon) such as a doctor can easily determine the magnitude of the dose in each of the plurality of angular regions partitioned at each predetermined angular interval of the imaging unit angle by visually confirming the displayed angular dose image 42, which enables the operator to easily determine the imaging unit angle with a relatively small dose. As a result, it is possible to easily select an appropriate imaging unit angle with a relatively low dose in order to suppress the localized increase in dose on the body surface of the subject P.

Further, in this embodiment, the following further effects can be obtained by configuring as follows.

In other words, in this embodiment, the dose calculation processing unit 70 includes the positional relation acquisition unit 71 that acquires a virtual positional relation between the virtual model Pa and the imaging unit 2 based on the positional relation between the table 1 and the imaging unit 2. The angle associating unit 73 (the dose calculation processing unit 70) is configured to update the association of the imaging unit angles on the surface of the virtual model Pa, based on the virtual positional relation acquired by the positional relation acquisition unit 71 when the positional relation between the table 1 and the imaging unit 2 is changed. The angular dose calculation unit 74 (the dose calculation processing unit 70) is configured to update the dose in each of the plurality of angular regions based on the association of the imaging unit angles on the surface of the updated virtual model Pa. With this configuration, even in the case where the positional relation between the table 1 and the imaging unit 2 is changed by moving the table 1, for example, the association of the imaging unit angles on the surface of the virtual model Pa is updated, so that the angular dose image 42 can be updated to accurately display the dose distribution.

As a result, even in the case where the positional relation between the table 1 and the imaging unit 2 is changed, the operator, such as a doctor, can easily and accurately select an appropriate imaging unit angle by visually observing the angular dose image 42.

Further, in this embodiment, the X-ray imaging system 100 is equipped with the touch panel 5 and the imaging operation unit 6 that accepts input operations by the operator (manipulator). The angular dose calculation unit 74 (the dose calculation processing unit 70) is configured to calculate the dose in each of the plurality of angular regions partitioned by each of the predetermined angular intervals whose size is changed when an operation to change the size of the predetermined angular interval that partitions the plurality of angular regions is received by the touch panel 5 or the operation unit 6. The monitor 4 (display unit) is configured to display the angular dose image 42 capable of identifying the magnitude of the dose in each of the plurality of angular regions in which the size of the predetermined angular interval is changed. By this configuration, an operator, such as a doctor, can change the size of the angular intervals of the plurality of angular regions in the angular dose image 42 by operating the touch panel 5 or the operation unit 6, and, therefore, can easily switch between checking the dose distribution in detail and checking the dose distribution as a whole.

Further, in this embodiment, the monitor 4 (display unit) is configured to display the angular dose image 42 in which a plurality of angular regions is partitioned in a grid pattern at each predetermined angular interval of the imaging unit angle, and the magnitude of the dose in each of the plurality of angular regions partitioned in a grid pattern can be identified. By configuring as described above, in the angular dose image 42, since a plurality of angular regions is partitioned in a grid pattern at predetermined angular intervals, the operator, such as the doctor, can easily recognize the dose distribution in two orthogonal angular directions of the subject P, the left-right directions (LAO and RAO) and the up-down directions (CRA and CAU). Therefore, the operator, such as the doctor, can easily determine which imaging unit angle should be changed in the two orthogonal angular directions of the subject P, the left-right directions (LAO and RAO) and the up-down directions (CRA and CAU), in order to suppress the localized increase in dose on the surface of the subject P's body.

Further, in this embodiment, the dose calculation processing unit 70 includes the storing processing unit 77 that stores the historical information on the dose distribution on the surface of the virtual model Pa, based on the X-ray irradiation by the imaging unit 2. The monitor 4 (display unit) is configured to display the angular dose image 42 at a predetermined timing in the past, based on the historical information stored by the storing processing unit 77. By configuring as described above, the operator, such as the doctor, can check the angular dose image 42 at a predetermined timing in the past, and, therefore, can easily check the progress of the dose increase to date. Therefore, in the case of selecting a new imaging unit angle for X-ray irradiation, the appropriate imaging unit angle can be selected more easily by referring to the progress of the dose increase to date.

Further, in this embodiment, the dose calculation processing unit 70 includes the dose prediction unit 78, which calculates the predicted value of the X-ray dose to be irradiated by the imaging unit 2, based on the predictive dose distribution on the surface of the virtual model Pa calculated by the model dose calculation unit 72 (the dose calculation processing unit 70). The monitor 4 (display unit) is configured to display the predicted angular dose image 42, based on the predicted value predicted by the dose prediction unit 78. By configuring as described above, it is possible for the operator, such as the doctor, to confirm the prediction unit on how the dose to the subject P will increase when X-ray irradiation is continued. Therefore, the operator can accurately determine whether to continue the X-ray irradiation with the current imaging unit angle remaining unchanged or to perform X-ray irradiation with the imaging unit angle changed. As a result, it is possible to effectively suppress the localized increase in dose on the body surface of the subject P.

Further, in this embodiment, the dose calculation processing unit 70 includes the irradiation region calculation unit 76 that calculates the irradiation region, which is a region of the surface of the virtual model Pa that is irradiated with X-rays at the current imaging unit angle. The monitor 4 (display unit) is configured to display a display (the irradiation region display 42a) that shows the angular regions included in the irradiation region from the plurality of angular regions in the angular dose image 42. By configuring as described above, by visually checking the angular dose image 42, it is possible to confirm which of the plurality of angular regions will increase in dose when X-ray irradiation is performed at the current imaging unit angle. Therefore, the operator, such as a doctor, can easily determine whether the dose to the subject P would become too large when X-rays are irradiated at the current imaging unit angle. As a result, the operators, such as a doctor, can easily determine whether or not to change the imaging unit angle in order to suppress the localized increase in dose on the body surface of the subject P.

Further, in this embodiment, the monitor 4 (display unit) is configured to display the maximum value display 44, separately from the angular dose image 42, so that the maximum value of the dose in the angular region included in the irradiation region can be identified. By configuring as described above, the operator, such as a doctor, can easily check the maximum dose at the current imaging unit angle. Accordingly, the operator, such as a doctor, can more easily determine whether or not to change the imaging unit angle.

Further, in this embodiment, the monitor 4 (display unit) is configured to display an angular region recommended for X-ray irradiation among the plurality of angular regions in the angular dose image 42 in an identifiable manner. By configuring as described above, it is possible for the operators, such as a doctor, to more easily determine which imaging unit angle is appropriate when changing the angle of the imaging unit. Therefore, when changing the imaging unit angle, the time and effort required to select a new imaging unit angle can be suppressed.

Further, in this embodiment, the moving mechanism 3 is configured to change the imaging unit angle so that the X-rays are irradiated to the selected angular region when an operation to select one of the plurality of angular regions in the angular dose image 42 (selection operation) is received by the operation unit 6. With this configuration, the operator, such as a doctor, can easily select a new imaging unit angle by performing an operation to select one of the plurality of angular regions in the angular dose image 42. Therefore, an operator, such as a doctor, can easily suppress the localized increase in dose on the body surface of the subject P by selecting an angular region with a relatively small dose from the plurality of angular regions in the angular dose image 42.

Further, in this embodiment, it is configured such that the monitor 4 (display unit) displays the angular dose image 42 in which the magnitude of the dose in each of a plurality of angular regions is represented by color-coding, and displays, separately from the angular dose image 42, a color scale image 43 indicating the color-coding of the plurality of angular regions in the angular dose image 42, which corresponds to the magnitude of the dose. By configuring as described above, the magnitude of the dose is displayed in the angular dose image 42 in color-coded manner, and the color scale image 43 showing the magnitude of the dose and the corresponding color-coding is also displayed. Therefore, the operator, such as a doctor, can more easily visually recognize the distribution of the magnitude of the dose in the angular dose image 42. Therefore, it is easy to recognize which of the angular regions has a larger dose, making it easier to select the appropriate imaging unit angle.

Further, in this embodiment, the monitor 4 (display unit) is configured to display preset dose thresholds 43a and 43b in the color scale image 43 in an identifiable manner. By configuring as described above, the operator, the preset dose thresholds 43a and 43b are displayed in the color scale image 43. Therefore, the operator, such as a doctor, can easily visually recognize which angular region's dose exceeds the preset dose thresholds 43a and 43b by checking the color-coding in the angular dose image 42. Therefore, by checking the angular dose image 42, the specific magnitude of the dose on the body surface of the subject P can be easily recognized.

Further, in this embodiment, the X-ray imaging system 100 is equipped with the touch panel 5, separately from the monitor 4 (display unit), which displays a plurality of imaging unit angles in a selectable manner and accepts an input operation to change the imaging unit angle. The touch panel 5 is configured to display the angular dose image 42. By configuring as described above, the operator, such as a doctor, can confirm the angular dose image 42 displayed on the touch panel 5 when changing the imaging unit angle by operating the touch panel 5. Therefore, as compared to checking the angular dose image 42 displayed on the monitor 4, which is separate from the touch panel 5, the viewpoint can be moved less, thus reducing the burden on the operator, such as the doctor, when selecting the imaging unit angle.

Further, in this embodiment, it is configured such that the monitor 4 (display unit) displays a three-dimensional image 46 of the subject P that has been acquired in advance and displays the angular region dose in the imaging unit angle corresponding to the display angle of the displayed three-dimensional image 46 in an identifiable manner on the angle indicator 46a. By configuring as described above, the dose in the angular region at the imaging unit angle corresponding to the display angle of the displayed three-dimensional image 46 is displayed on the angle indicator 46a in an identifiable manner. Therefore, the operator, such as a doctor, can more easily select an imaging unit angle with a relatively small dose when selecting an appropriate imaging unit angle while viewing the three-dimensional image 46.

In the dose display method of this embodiment, by configuring as described above, the dose in each of a plurality of imaging angles partitioned for each predetermined angular interval of the imaging unit angle is calculated based on the calculated dose distribution on the surface of the virtual model Pa and the imaging unit angles associated with the surface of the virtual model Pa. The X-ray imaging system 100 displays the angular dose image 42, which is capable of identifying the magnitude of the dose in each of the plurality of angular regions. With this, an operator (surgeon) such as a doctor can easily determine the magnitude of the dose in each of the plurality of angular regions partitioned at each predetermined angular interval of the imaging unit angle by visually confirming the displayed angular dose image 42, which enables the operator to easily determine the imaging unit angle with a relatively small dose. As a result, it is possible to provide a dose display method capable of easily selecting an appropriate imaging unit angle with a relatively low dose in order to suppress the localized increase in dose on the body surface of the subject P.

### Modifications

Note that the embodiments disclosed here should be considered illustrative and not restrictive in all respects. It should be noted that the scope of the invention is indicated by claims and is intended to include all modifications (modified examples) within the meaning and scope of the claims and equivalents.

For example, in the above embodiment, an example is shown in which the imaging unit 2 has a single-plane mechanism with one X-ray irradiation unit 21 and one X-ray detection unit 22, but the present invention is not limited thereto. In the present invention, the imaging unit 2 may be configured to be equipped with a biplane mechanism with two X-ray irradiation units 21 and two X-ray detection units 22. In that case, in the angular dose image 42, two irradiation regions (irradiation region display 42a) are displayed to correspond to the two X-ray irradiation units 21.

Further, in the above-described embodiment, an example is shown in which when the positional relation between the table 1 and the imaging unit 2 is changed, the association of the imaging unit angles on the surface of the virtual model Pa is updated. For example, even in cases where the positional relation between the table 1 and the imaging unit 2 is not changed, the system may be configured to update the association of the imaging unit angles on the surface of the virtual model Pa each time the imaging unit angle is changed.

Further, in the above-described embodiment, an example is shown in which the size of the predetermined angular intervals partitioning the plurality of angular regions is changed based on input operations to the touch panel 5 and the operation unit 6. For example, it may be configured such that the calculated dose distribution on the surface of the virtual model Pa is displayed as it is in the angular dose image 42 in a state of being associated with the imaging unit angle. In other words, in the angular dose image 42, the dose distribution may be displayed smoothly (in more detail) based on the dose calculated for each micro region in the virtual model Pa, without partitioning the angular region in a grid pattern.

Further, in the above-described embodiment, an example is shown in which the angular dose image 42 at each timing in which the X-ray image 41 as a moving image is captured is displayed as the angular dose image 42 at a predetermined timing in the past, but the present invention is not limited thereto. For example, the angular dose image 42 at each predetermined time interval may be displayed on the monitor 4 as the angular dose image 42 at a predetermined timing in the past.

In the above-described embodiment, an example is shown in which an imaging angular dose image 42 that is predicted when X-ray irradiation is continued in the irradiation region at the current imaging unit angle is displayed, but the present invention is not limited thereto. For example, it may be configured to display an imaging angular dose image 42 that is predicted when the imaging unit angle is changed.

Further, in the above-described embodiment, an example is shown in which the irradiation region (the irradiation region display 42a) is also displayed in the angular dose image 42, but the present invention is not limited thereto. For example, the irradiation region may not be displayed on the angular dose image 42. Further, instead of showing the irradiation region so as to enclose the entire angular region including the irradiation region, the irradiation region may be shown by means of a more detailed display range, separate from the partition of each angular region.

Further, in the above-described embodiment, an example is shown in which the maximum value (the maximum value display 44) of the dose in the angular region included in the irradiation region is displayed, but the present invention is not limited thereto. For example, the average value of the dose in the angular region included in the irradiation region may be displayed.

Further, in the above-described embodiment, an example is shown in which the recommended angular regions are color-coded with a predetermined color to indicate the recommended angular regions in the angular dose image 42, but the present invention is not limited thereto. For example, it may be shown to show the recommended angular region in the angular dose image 42 by color-coding to surround the recommended angular region.

Further, in the above-described embodiment, an example is shown in which one angular region including the imaging unit angle for performing the next X-ray irradiation is selected from a plurality of angular regions in the angular dose image 42 displayed on the monitor 4 (display unit), but the present invention is not limited. For example, one angular region may be selected from the plurality of angular regions in the imaging angular dose image 42 displayed on the touch panel 5, including the imaging unit angle at which the next X-ray irradiation is to be performed.

Further, in the above-described embodiment, an example is shown in which the angular dose image 42 and the color scale image 43 are displayed side by side on the monitor 4 (display unit), but the present invention is not limited thereto. For example, the color scale image 43 may be temporarily displayed based on the input operation to the touch panel 5 and the operation unit 6. Similarly, the thresholds 43a and 43b in the color scale image 43 may be temporarily displayed based on input operations.

Further, in the above-described embodiment, an example is shown in which the angular dose image 42 is displayed on the touch panel 5 by switching the display based on an input operation, but the present invention is not limited thereto. For example, it may be configured to display the plurality of imaging unit angle display 5a and the angular dose image 42.

Further, in the above-described embodiment, an example is shown in which the X-ray image 41, the angular dose image 42, and the three-dimensional image 46 are displayed on the monitor 4 (display unit), but the present invention is not limited thereto. For example, the X-ray image 41 may be displayed on a display device provided separately from the monitor 4. Further, the three-dimensional image 46 may be displayed on a display device, separately from the monitor 4.

Further, in the above-described embodiment, an example is shown in which the control device 101 controls X-ray imaging, the dose calculation device 102 that calculates the dose, and the three-dimensional image generation device 103 that generates the three-dimensional image 46 are each provided as separate devices, but the present invention is not limited thereto. For example, any two or more of the control of X-ray imaging, the calculation of the dose, and the generation of the three-dimensional image 46 may be performed by a common processing device.

Further, in the above-described embodiment, an example is shown in which the control processing in the X-ray imaging system 100 is described using a flow-driven flowchart in which processing is performed in sequence according to a processing flow, but the present invention is not limited thereto. In the present invention, the control processing in the X-ray imaging system 100 may be performed by event-driven processing that executes processing on an event-by-event basis. In this case, it may be performed in a completely event-driven manner, or a combination of event-driven and flow-driven.

### Aspects

It would be understood by those skilled in the art that the exemplary embodiments described above are specific examples of the following aspects.

### Item 1

An X-ray imaging system comprising:
an imaging unit including an X-ray irradiation unit having an X-ray source that irradiates a subject on a table with X-rays and an X-ray detection unit that detects X-rays emitted from the X-ray irradiation unit;
a moving mechanism configured to change an imaging unit angle which is an angle of the imaging unit;
a dose calculation processing unit configured to calculate a dose of X-rays irradiated onto the subject; and
a display unit configured to display the dose of X-rays calculated by the dose calculation processing unit,
wherein the dose calculation processing unit includes
a model dose calculation unit configured to calculate a dose distribution on a surface of a three-dimensional virtual model representing the subject on the table, based on the dose of the X-rays irradiated onto the subject by the imaging unit,
an angle associating unit configured to associate the imaging unit angle with a surface of the virtual model, based on the imaging unit angle and a position on the surface of the virtual model to be irradiated with X-rays, and,
an angular dose calculation unit configured to calculate the dose in each of a plurality of angular regions partitioned by each of predetermined angular intervals of the imaging unit angle, based on the dose distribution on the surface of the virtual model calculated by the model dose calculation unit and the imaging unit angle associated with the surface of the virtual model by the angle associating unit, and
wherein the display unit is configured to display an angular dose image capable of identifying magnitude of the dose in each of the plurality of angular regions calculated by the angular dose calculation unit.

### Item 2

The X-ray imaging system according to the above-described Item 1,
wherein the dose calculation processing unit further includes a positional relation acquisition unit that acquires a virtual positional relation between the virtual model and the imaging unit, based on a positional relation between the table and the imaging unit,
wherein the angle associating unit is configured to update an association of the imaging unit angle on the surface of the virtual model, based on the virtual positional relation acquired by the positional relation acquisition unit, when the positional relation between the table and the imaging unit is changed, and
wherein the angular dose calculation unit is configured to update the dose in each of the plurality of angular regions, based on the association of the imaging unit angle on the surface of the updated virtual model.

### Item 3

The X-ray imaging system according to the above-described Item 1 or 2, further comprising:
an operation unit configured to accept an input operation by an operator,
wherein the angular dose calculation unit is configured to calculate the dose in each of the plurality of angular regions partitioned by each of the predetermined angular intervals with a modified size, when an operation to change a size of each of the predetermined angular intervals partitioning the plurality of angular regions is accepted by the operation unit, and
wherein the display unit is configured to display the angular dose image capable of identifying the magnitude of the dose in each of the plurality of angular regions with the size of the predetermined angular interval changed.

### Item 4

The X-ray imaging system according to any one of the above-described Items 1 to 3,
wherein the display unit is configured to display the angular dose image in which the plurality of angular regions is partitioned by each of the predetermined angular intervals of the imaging unit angle in a grid pattern, and the magnitude of the dose in each of the plurality of angular regions partitioned in a grid pattern is capable of being identified.

### Item 5

The X-ray imaging system according to any one of the above-described Items 1 to 4,
wherein the dose calculation processing unit further includes a storing processing unit that stores historical information on the dose distribution on the surface of the virtual model, based on X-ray irradiation by the imaging unit, and
wherein the display unit is configured to display the angular dose image at a predetermined timing in the past, based on the historical information stored by the storing processing unit.

### Item 6

The X-ray imaging system according to any one of claims 1 to 5,
wherein the dose calculation processing unit further includes a dose prediction unit that calculates a predicted value for the dose of X-rays to be irradiated by the imaging unit, based on the dose distribution on the surface of the virtual model calculated by the model dose calculation unit, and
wherein the display unit is configured to display the predicted angular dose image based on the predicted value predicted by the dose prediction unit.

### Item 7

The X-ray imaging system according to any one of the above-described Items 1 to 6, on unit that calculates an irradiation region which is a region of the surface of the virtual model to be irradiated with X-rays at a current imaging unit angle, and
wherein the display unit is configured to display an indication of the angular region that is included in the irradiation region from the plurality of angular regions in the angular dose image.

### Item 8

The X-ray imaging system according to the above-described Item 7,
wherein the display unit is configured to display, separately from the angular dose image, a maximum value of the dose in the angular region included in the irradiation region in an identifiable manner.

### Item 9

The X-ray imaging system according to any one of the above-described Items 1 to 8,
wherein the display unit is configured to display the angular region in which X-ray irradiation is recommended among the plurality of angular regions in the angular dose image in an identifiable manner.

### Item 10

The X-ray imaging system according to any one of the above-described Items 1 to 9, further comprising:
an operation unit configured to accept an input operation by an operator,
wherein the moving mechanism is configured to change the imaging unit angle so that the selected angular region is irradiated with X-rays when an operation to select one of the plurality of angular regions in the angular dose image is accepted by the operation unit.

### Item 11

The X-ray imaging system according to any one of the above-described Items 1 to 10,
wherein the display unit is configured
to display the angular dose image in which the magnitude of the dose in each of the plurality of angular regions is color-coded, and
to display, separately from the angular dose image, a color scale image showing the color-coding of the plurality of angular regions in the angular dose image corresponding to the magnitude of the dose.

### Item 12

The X-ray imaging system according to the above-described Item 11,
wherein the display unit is configured to display a preset dose threshold in the color scale image in an identifiable manner.

### Item 13

The X-ray imaging system according to any one of the above-described Items 1 to 12, further comprising, in addition to the display unit:
a touch panel configured to display the plurality of imaging unit angles in a selectable manner and accept an input operation for changing the imaging unit angle,
   wherein the touch panel is configured to display the angular dose image.

### Item 14

The X-ray imaging system according to any one of the above-described Items 1 to 13,
wherein the display unit is configured
to display a pre-acquired three-dimensional image of an inside of the subject, and
to display the dose of the angular region at the imaging unit angle corresponding to a display angle of the displayed three-dimensional image in an identifiable manner.

### Item 15

A dose display method comprising:
a step of calculating a dose distribution on a surface of a three-dimensional virtual model representing a subject on a table, based on the dose of X-rays irradiated onto the subject by X-ray irradiation by an imaging unit, the imaging unit including an X-ray irradiation unit having an X-ray source that irradiates the subject on the table with X-rays and an X-ray detection unit that detects the X-rays irradiated from the X-ray irradiation unit;
a step of associating an imaging unit angle with a surface of the virtual model, based on the imaging unit angle that is an angle of the imaging unit and a position on a surface of the virtual model to be irradiated with the X-rays;
a step of calculating the dose in each of a plurality of angular regions partitioned by each of predetermined angular intervals of the imaging unit angle, based on the calculated dose distribution on the surface of the virtual model and the imaging unit angle associated with the surface of the virtual model; and
a step of displaying an angular dose image capable of identifying magnitude of the calculated dose in each of the plurality of angular regions.

### DESCRIPTION OF REFERENCE SYMBOLS

1: Table
2: Imaging unit
3: Moving mechanism
4: Monitor (Display unit)
5: Touch panel (Operation unit)
6: Operation unit
21: X-ray irradiation unit
21a: X-ray source
22: X-ray detection unit
42: Angular dose image
43: Color scale image
43a, 43b: Threshold
46: Three-dimensional image
70: Dose calculation processing unit
71: Positional relation acquisition unit
72: Model dose calculation unit
73: Angle associating unit
74: Angular dose calculation unit
76: Irradiation region calculation unit
77: Storing processing unit
78: Dose prediction unit
100: X-ray imaging system

1. An X-ray imaging system comprising:
an imaging unit including an X-ray irradiation unit having an X-ray source that irradiates a subject on a table with X-rays and an X-ray detection unit that detects X-rays emitted from the X-ray irradiation unit;
a moving mechanism configured to change an imaging unit angle which is an angle of the imaging unit;
a dose calculation processing unit configured to calculate a dose of X-rays irradiated onto the subject; and
a display unit configured to display the dose of X-rays calculated by the dose calculation processing unit,
wherein the dose calculation processing unit includes
a model dose calculation unit configured to calculate a dose distribution on a surface of a three-dimensional virtual model representing the subject on the table, based on the dose of the X-rays irradiated onto the subject by the imaging unit,
an angle associating unit configured to associate the imaging unit angle with a surface of the virtual model, based on the imaging unit angle and a position on the surface of the virtual model to be irradiated with X-rays, and,
an angular dose calculation unit configured to calculate the dose in each of a plurality of angular regions partitioned by each of predetermined angular intervals of the imaging unit angle, based on the dose distribution on the surface of the virtual model calculated by the model dose calculation unit and the imaging unit angle associated with the surface of the virtual model by the angle associating unit,
wherein the display unit is configured to display an angular dose image capable of identifying magnitude of the dose in each of the plurality of angular regions calculated by the angular dose calculation unit, and
wherein the display unit is configured to display the angular region in which X-ray irradiation is recommended among the plurality of angular regions in the angular dose image in an identifiable manner.

2. The X-ray imaging system according to claim 1,
wherein the dose calculation processing unit further includes a positional relation acquisition unit that acquires a virtual positional relation between the virtual model and the imaging unit, based on a positional relation between the table and the imaging unit,
wherein the angle associating unit is configured to update an association of the imaging unit angle on the surface of the virtual model, based on the virtual positional relation acquired by the positional relation acquisition unit, when the positional relation between the table and the imaging unit is changed, and
wherein the angular dose calculation unit is configured to update the dose in each of the plurality of angular regions, based on the association of the imaging unit angle on the surface of the updated virtual model.

3. The X-ray imaging system according to claim 1, further comprising:
an operation unit configured to accept an input operation by an operator,
wherein the angular dose calculation unit is configured to calculate the dose in each of the plurality of angular regions partitioned by each of the predetermined angular intervals with a modified size, when an operation to change a size of each of the predetermined angular intervals partitioning the plurality of angular regions is accepted by the operation unit, and
wherein the display unit is configured to display the angular dose image capable of identifying the magnitude of the dose in each of the plurality of angular regions with the size of the predetermined angular interval changed.

4. The X-ray imaging system according to claim 1,
wherein the display unit is configured to display the angular dose image in which the plurality of angular regions is partitioned by each of the predetermined angular intervals of the imaging unit angle in a grid pattern, and the magnitude of the dose in each of the plurality of angular regions partitioned in a grid pattern is capable of being identified.

5. The X-ray imaging system according to claim 1,
wherein the dose calculation processing unit further includes a storing processing unit that stores historical information on the dose distribution on the surface of the virtual model, based on X-ray irradiation by the imaging unit, and
wherein the display unit is configured to display the angular dose image at a predetermined timing in the past, based on the historical information stored by the storing processing unit.

6. The X-ray imaging system according to claim 1,
wherein the dose calculation processing unit further includes a dose prediction unit that calculates a predicted value for the dose of X-rays to be irradiated by the imaging unit, based on the dose distribution on the surface of the virtual model calculated by the model dose calculation unit, and
wherein the display unit is configured to display the predicted angular dose image based on the predicted value predicted by the dose prediction unit.

7. The X-ray imaging system according to claim 1,
wherein the dose calculation processing unit further includes an irradiation region calculation unit that calculates an irradiation region which is a region of the surface of the virtual model to be irradiated with X-rays at a current imaging unit angle, and
wherein the display unit is configured to display an indication of the angular region that is included in the irradiation region from the plurality of angular regions in the angular dose image.

8. The X-ray imaging system according to claim 7,
wherein the display unit is configured to display, separately from the angular dose image, a maximum value of the dose in the angular region included in the irradiation region in an identifiable manner.

9. (canceled)

10. An X-ray imaging system comprising:
an imaging unit including an X-ray irradiation unit having an X-ray source that irradiates a subject on a table with X-rays and an X-ray detection unit that detects X-rays emitted from the X-ray irradiation unit;
a moving mechanism configured to change an imaging unit angle which is an angle of the imaging unit;
a dose calculation processing unit configured to calculate a dose of X-rays irradiated onto the subject; and
a display unit configured to display the dose of X-rays calculated by the dose calculation processing unit,
wherein the dose calculation processing unit includes
a model dose calculation unit configured to calculate a dose distribution on a surface of a three-dimensional virtual model representing the subject on the table, based on the dose of the X-rays irradiated onto the subject by the imaging unit,
an angle associating unit configured to associate the imaging unit angle with a surface of the virtual model, based on the imaging unit angle and a position on the surface of the virtual model to be irradiated with X-rays, and,
an angular dose calculation unit configured to calculate the dose in each of a plurality of angular regions partitioned by each of predetermined angular intervals of the imaging unit angle, based on the dose distribution on the surface of the virtual model calculated by the model dose calculation unit and the imaging unit angle associated with the surface of the virtual model by the angle associating unit,
wherein the display unit is configured to display an angular dose image capable of identifying magnitude of the dose in each of the plurality of angular regions calculated by the angular dose calculation unit, and
wherein the X-ray imaging system further comprises:
an operation unit configured to accept an input operation by an operator, and
wherein the moving mechanism is configured to change the imaging unit angle so that the selected angular region is irradiated with X-rays when an operation to select one of the plurality of angular regions in the angular dose image is accepted by the operation unit.

11. The X-ray imaging system according to claim 1,
wherein the display unit is configured
to display the angular dose image in which the magnitude of the dose in each of the plurality of angular regions is color-coded, and
to display, separately from the angular dose image, a color scale image showing the color-coding of the plurality of angular regions in the angular dose image corresponding to the magnitude of the dose.

12. The X-ray imaging system according to claim 11,
wherein the display unit is configured to display a preset dose threshold in the color scale image in an identifiable manner.

13. The X-ray imaging system according to claim 1, further comprising, in addition to the display unit:
a touch panel configured to display the plurality of imaging unit angles in a selectable manner and accept an input operation for changing the imaging unit angle,
wherein the touch panel is configured to display the angular dose image.

14. The X-ray imaging system according to claim 1,
wherein the display unit is configured
to display a pre-acquired three-dimensional image of an inside of the subject, and
to display the dose of the angular region at the imaging unit angle corresponding to a display angle of the displayed three-dimensional image in an identifiable manner.

15. A dose display method comprising:
a step of calculating a dose distribution on a surface of a three-dimensional virtual model representing a subject on a table, based on the dose of X-rays irradiated onto the subject by X-ray irradiation by an imaging unit, the imaging unit including an X-ray irradiation unit having an X-ray source that irradiates the subject on the table with X-rays and an X-ray detection unit that detects the X-rays irradiated from the X-ray irradiation unit;
a step of associating an imaging unit angle with a surface of the virtual model, based on the imaging unit angle that is an angle of the imaging unit and a position on a surface of the virtual model to be irradiated with the X-rays;
a step of calculating the dose in each of a plurality of angular regions partitioned by each of predetermined angular intervals of the imaging unit angle, based on the calculated dose distribution on the surface of the virtual model and the imaging unit angle associated with the surface of the virtual model; and
a step of displaying an angular dose image capable of identifying magnitude of the calculated dose in each of the plurality of angular regions.
